(19) United States
(12) Patent Application Publication
(10) Pub. No.: US 2015/0065788 A1
(43) Pub. Date: Mar. 5, 2015

(54) INCUBATOR WITH DOUBLE GLAZED WALL AND METHODS THEREOF

(71) Applicant: ASPECT IMAGING LTD., Shoham (IL)

(72) Inventor: Uri RAPOPORT, Moshav Ben Shemen (IL)

(21) Appl. No.: 14/471,015

(22) Filed: Aug. 28, 2014

Related U.S. Application Data
(60) Provisional application No. 61/872,789, filed on Sep. 2, 2013.

(30) Foreign Application Priority Data
Dec. 23, 2013 (DE) 20 2013 105 901.3

(51) Int. Cl. A61G 11/00; A61M 21/00; A61M 21/02; A61F 7/00

(52) U.S. Cl. CPC A61G 11/00; A61F 7/0053; A61F 7/007; A61M 21/0094; A61M 21/02; A61G 2203/70; A61F 2007/0075; A61F 2007/0088; A61F 2007/0054; A61M 2021/0027; A61M 2021/0066; A61M 2205/052; A61M 2205/42; A61M 2240/00; A61M 2205/02
USPC 600/22

(57) ABSTRACT

An incubator with a double glazed wall that promotes regulation of sound pressure levels and temperature levels the incubator, using an insulated glass unit. The canopy of the incubator has at least one insulated glass unit. This unit includes at least one first inner glass and at least one second external glass. The inner and external glasses are spaced by a spacer, and are characterized by an inner atmosphere confined within the canopy and an external atmosphere.

### FIELD OF THE INVENTION

The present invention generally relates to incubator with a double glazed wall. More specifically, the invention related to means and method for avoiding sound pressure levels in and increasing temperature regulation in infant's incubator using an insulated glass unit and the methods of achieving thereof.

### BACKGROUND OF THE INVENTION

High sound pressure levels may be harmful to the maturing newborn. Current guidelines suggest that the sound pressure levels within a neonatal intensive care unit should not exceed 45 dB. It is likely that environmental noise as well as the noise generated by the incubator fan and respiratory equipment may contribute to the total sound pressure levels. Knowledge of the contribution of each component and source is important to develop effective strategies to reduce noise within the incubator. It is widely accepted that the sound levels, especially at low frequencies, within a modern incubator may reach levels that are likely to be harmful to the developing newborn. Much of the noise is at low frequencies and thus difficult to reduce by conventional means. Therefore, advanced forms of noise control are needed to address this issue, see for example Marik et al., Neonatal incubators: a toxic sound environment for the preterm infant? Pediatr Crit Care Med. 2012 November; 13(6):685-9, which is incorporated herein as a reference.

More than that, one of the most important elements in a newborn's survival is the infants temperature regulation, see e.g., currently available link http://www.ebme.co.uk/. Mammals have the advantage of being homeotherms, meaning that they are able to produce heat, allowing us to maintain a constant body temperature. However, homeothermy may be overwhelmed in extremes of cold or heat. The newborn baby has all the capabilities of a mature homeotherm, but the range of environmental temperature over which an infant can operate successfully is severely restricted. The infant's body responds differently to hot and cold temperatures. In the case of hotter environmental temperatures, the infant's body produces sweat through the sweat glands.

The basal metabolic rate increases, causing the body temperature to rise. The risks of hyperthermia are great and should be attended to immediately. Serious overheating can cause heatstroke or death, and lesser degrees of stress can cause cerebral damage due to hypernatremic dehydration. Babies born more than 8 weeks before term have virtually no ability to sweat. Even in a baby born only 3 weeks early, sweating is severely limited and confined to the head and face. Sweat production matures relatively quickly in the pre-term baby after delivery, allowing the baby to be placed in a regular crib. In the case of cold environmental temperatures, the infant may produce heat by shivering and other muscular activity. Cold stress is subtler in its consequences but must be attended to. Newborns may also be placed in a neonatal incubator. Hence, increased thermal stability in the incubator on air mode control may well be beneficial, particularly to sick, very low birthweight infants; See Ducker et al., Incubator temperature control: effects on the very low birthweight infant. Arch Dis Child. 1985 October; 60(10): 902-907 which is incorporated herein as a reference.

Various applicable standards and guidelines are incorporated herein as a reference, including IEC 601-2-19 Amendment I Medical Electrical Equipment—Part 2: Particular Requirements for Safety of Baby Incubators; IEC 601-2-20 Medical Electrical Equipment—Part 2: Particular Requirements for Safety of Transport Incubators; ISO 10993-1: 1992 Biological Evaluation of Medical Devices—Part 1: Evaluation and Testing; AAMI 1151-D-1994 Transport Infant Incubator (Draft Standard); AAMI/CDV-1 II36 1997 Infant Incubator (Draft Standard).

It is thus a long felt need to provide integrated safe means for avoiding high sound pressure levels and increase temperature regulation and the methods of achieving thereof.

### SUMMARY OF THE INVENTION

It is an object of the invention to disclose an incubator with a canopy. The canopy is essentially having at least one insulated glass unit (IGU, 10). This unit comprising at least one first inner glass (1a) and at least one second external glass (1b), said inner and external glasses are spaced (2) by a spacer (3), characterized by an inner atmosphere (21) confined within said canopy and an external atmosphere (22); wherein said IGU both (i) avoids sound pressure levels in said inner atmosphere from exceeding 45 dB, and (ii) increase temperature regulation in said inner atmosphere, such that temperature interval in not greater than 1° C. for air controlled transport incubators and not greater than 0.5° C. for baby controlled transport incubators.

It is an object of the invention to disclose an incubator comprising active and passive noise reduction and temperature regulation mechanisms selected from a group consisting of: passive sound and thermal isolator environment characterized by specific thickness of said inner glass, thickness of said external glass; and by means of specific gas inserted in said atmosphere within said inner and external glasses; passive sound and thermal isolator environment characterized by specific thickness of said inner glass, thickness of said external glass; and by means of vacuum in said atmosphere within said inner and external glasses; a reactive acoustical device, configured to cancel said device's surrounding acoustical noise by destructive interference; an active acoustical sound-speaker, located adjacent to said patient and/or adjacent to said noise generator; said sound speaker emits white acoustical noise such said noise is filtered and masked; and an active temperature control mechanism, configured to maintain a specific temperature within said incubator.

It is an object of the invention to disclose an incubator as defined above, wherein said IGU comprising a gas space of 16-19 mm when measured at the center of the IGU.

It is an object of the invention to disclose an incubator as defined above, wherein said IGU is characterized by an overall thickness of the IGU of 22-25 mm for 3 mm glass to 28-31 mm for 6.35 mm plate glass.

It is an object of the invention to disclose an incubator wherein, said spacers are made of materials selected from a group consisting of: metal, fiber, structural foam, aluminum, PVC, wood, and any combination thereof.

It is an object of the invention to disclose an incubator, wherein said spacers are filled with or contain desiccant to remove moisture.

It is an object of the invention to disclose an incubator, wherein said atmosphere between said first and second glasses is filled with gases selected from a group consisting of: argon, krypton, xenon, sulfur hexafluoride and any combination thereof.

It is an object of the invention to disclose an incubator as defined above, wherein said IGU is a Vacuum Insulated Glass (VIG) comprising an evacuating gap.

It is an object of the invention to disclose an incubator as defined above, wherein said IGU VIG is characterized by U-values for walls of about 0.3 to 1.2 W/(m²K); the term ‘about’ refers hereinafter to a value being 25% lower or greater than the defined measure.

It is an object of the invention to disclose a sound dampening incubator as defined in any of the above, wherein said IGU is selected from a group consisting of laminated glass, IGU, VIG and a combination thereof.

It is an object of the invention to disclose a sound dampening incubator as defined in any of the above, wherein the IGU is characterized by a laminate which reduces the ‘coincidence dip’ attributed to monolithic glass in the 1000-2000 Hz range.

It is an object of the invention to disclose an incubator, wherein said temperature control mechanism consisting by a group consisting of: air conditioned system, an infrared heater, a water in oil-heated radiator, a coiled heater, an open coil air heater, a round open coil air heater, a convection heater, straight or formed tubular heaters, a quartz tube air heater, a capacitor-type heater, a Pelletier module.

It is an object of the invention to disclose a method of avoiding sound pressure levels in an inner atmosphere of an incubator from exceeding 45 dB, and increasing temperature regulation in said inner atmosphere, such that temperature interval in not greater than 1° C. for air controlled transport incubators and not greater than 0.5° C. for baby controlled transport incubators, comprising a step of providing an incubator with a canopy, and further providing said canopy with at least one insulated glass unit (IGU, 10), by providing the same with at least one first inner glass (1a) and at least one second external glass (1b), and spacing said inner and external glasses by a spacer (3).

It is an object of the invention to disclose a method as defined in any of the above, wherein said method further comprising step of providing said IGU with a gas space of 16-19 mm when measured at the center of the IGU.

It is an object of the invention to disclose a method as defined in any of the above, wherein said method further comprising step of providing said IGU with an overall thickness of 22-25 mm for 3 mm glass to 28-31 mm for 6.35 mm plate glass.

It is an object of the invention to disclose a method as defined in any of the above, wherein said method further comprising step of providing said spacers made of materials selected from a group consisting of: metal, fiber, structural foam, aluminum, PVC, wood, and any combination thereof.

It is an object of the invention to disclose a method as defined in any of the above, wherein said method further comprising step of providing said spacers filled with or contain desiccant to remove moisture.

It is an object of the invention to disclose a method as defined in any of the above, wherein said method further comprising step of providing said atmosphere within said first and second glasses is filled with gases selected from a group consisting of: argon, krypton, xenon, sulfur hexafluoride and any combination thereof.

It is an object of the invention to disclose a method as defined in any of the above, wherein said method further comprising step of providing said IGU with Vacuum Insulated Glass (VIG) comprising an evacuating gap.

It is an object of the invention to disclose a method as defined in any of the above, wherein said VIG is characterized by U-values for walls of about 0.3 to 1.2 W/(m²K).

It is an object of the invention to disclose a method of sound dampening in an incubator, comprising a step of selecting said IGU from a group consisting of laminated glass, IGU, VIG and a combination thereof.

It is an object of the invention to disclose a method of sound dampening in an incubator, as defined above, wherein said method comprising step of reducing the ‘coincidence dip’ attributed to monolithic glass in the 1000-2000 Hz range.

It is an object of the invention to disclose a method as defined in any of the above, wherein said method further comprising step of providing said temperature control mechanism consisting by a group consisting of: air conditioned system, an infrared heater, a water/oil-heated radiator, a coiled heater, an open coil air heater, a round open coil air heater, a convection heater, straight or formed tubular heaters, a quartz tube air heater, a capacitor-type heater, a Pelletier module.

### BRIEF DESCRIPTION OF THE FIGURES

In order to understand the invention and to see how it may be implemented in practice, a few preferred embodiments will now be described, by way of non-limiting example only, with reference to be accompanying drawings, in which:

FIG. 1 schematically illustrates an IGU (10) according to an embodiment of the invention;

FIG. 2 illustrates an IGU (10), incorporated as a canopy's wall, namely an infant's incubator (20) comprising a canopy according to an embodiment of the invention;

FIG. 3 schematically illustrates a Vacuum Insulated Glass according to an embodiment of the invention; and

FIG. 4 schematically illustrates a laminated glass from incubator's canopy.

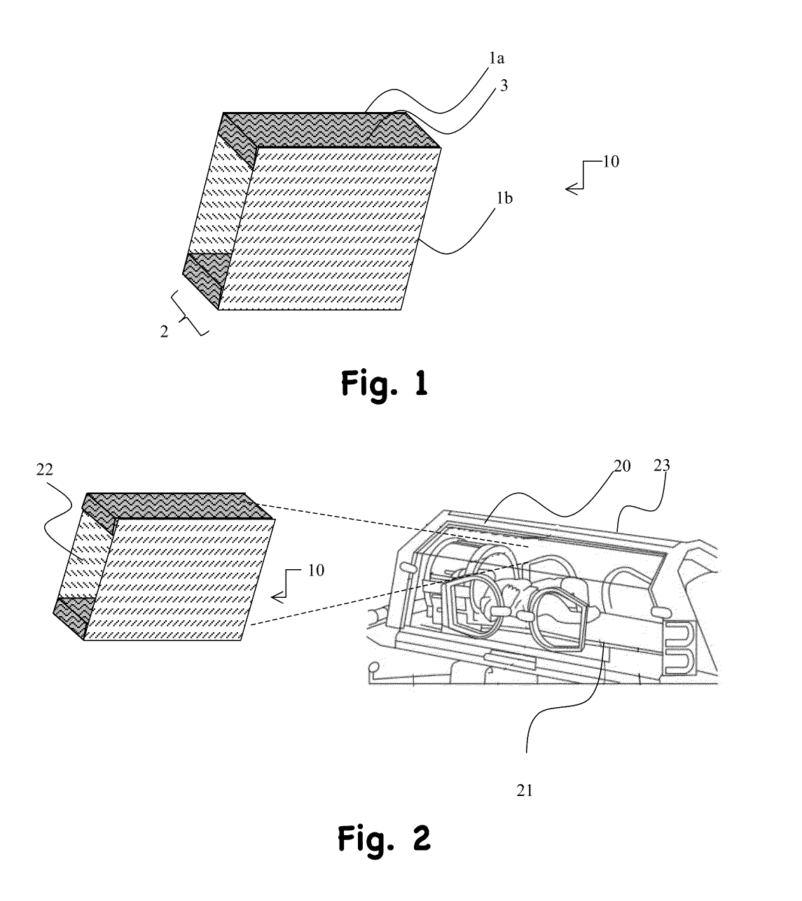

### DETAILED DESCRIPTION OF PREFERRED EMBODIMENTS

The term “human hearing” interchangeably refers herein to any sound received by the human ear, with the typical frequency range for normal hearing being between 20-Hz to 20,000-Hz. The logarithmic decibel scale, dB, is used when referring to sound power.

The term “noise” interchangeably refers herein to any unwanted sound defined in terms of frequency spectrum (in Hz), intensity (in dB), and time duration. Noise can be steady-state, intermittent, impulsive, or explosive. Transient hearing loss may occur following exposure to loud noise, resulting in a temporary threshold shift (i.e., a shift in the audible threshold). This term further includes harmonious and/or non-harmonious sounds, intended and/or unintended such as: a melody, tapping, banging, chirping, squeaking, blast, buzz, cacophony, clamor, commotion, crash, echo, cry, explosion, roar, babel, bang, bellow, blare, boom, caterwauling, clang, clatter, detonation, din, discord, disquiet, disquietude, drumming, eruption, jangle, lamentation, outcry, pandemonium, peal, racket, knocking, shot, shouting, squawk, stridency, thud, uproar, yell, music, or any combination thereof including a single or plurality of each.

Noise tends to be enhanced by decreases in section thickness, field of view, repetition time, and echo time Furthermore, noise characteristics have a spatial dependence. For example, noise levels can vary by as much as 10 dB as a function of patient position within a defined space such as the inside of an incubator. The presence and size of the patient may also affect the level of acoustic noise. Airborne sound travels through the air and can transmit through a material, assembly or partition. Sound can also pass under doorways, through ventilation, over, under, around, and through obstructions. When sound reaches a room where it is unwanted, it becomes noise. Further noise can be prolonged and multiplied by reverberations and reflections.

The term “sound attenuation means” interchangeably refers herein to any means configured for attenuating or muffling general and specific sounds, including noise. These means include passive acoustic attenuators such as resonators designed for specific frequencies, sound absorptive materials and linings, sound shields, bass traps, diffusers, sound baffles, and any combination thereof. Passive sound absorptive materials that are used incorporated with the medical device, having at least a portion of the sound energy dissipated within the medium itself as sound travels through them can be such as porous materials commonly formed of matted or spun fibers; panel (membrane) absorbers having an impervious surface mounted over an airspace; and resonators created by holes or slots connected to an enclosed volume of trapped air. Common porous absorbers allow air to flow into a cellular structure where sound energy is converted to heat. These may include a thick layer of cloth or carpet, spray-applied cellulose, aerated plaster, fibrous mineral wool and glass fiber, open-cell foam, and felted or cast porous ceiling tile. Further any acoustic insulation materials can be employed. Thickness plays an important role in sound absorption by porous materials.

Other absorbers are panel absorbers. Typically, panel absorbers are non-rigid, non-porous materials which are placed over an airspace that vibrates in a flexural mode in response to sound pressure exerted by adjacent air molecules for example thin wood paneling over framing, lightweight impervious ceilings and floors, glazing and other large surfaces capable of resonating in response to sound.

The term “Acoustic insulation material” interchangeably refers herein to any material with the ability to absorb sound, act as a barrier of sound, or both. This can refer in a non-limiting manner to materials such as: cork, wool, cotton, Eel grass, fiber glass, glass wool, wood, paper, Cobalt Quilt, sugarcane, hydrated Calcium sulphate, POP, Coir, plastic, PVC, perforated metal, Mineral fiber board, or Micore, Thermocole, Polyurethane, Jute, Mylar film, melamine, rubber, rock wool, cellulose, polystyrene, polyethylene, polyester, metal any of these materials when recycled, and etc. Further the acoustic material can be in one or more forms such as a sheet, fabric, tile, blanket, foam, rug, carpet, drape, curtain, panel, board, any casted shape, rod, block, beads, straw like, gravel like particles, Fabric can be wrapped around substrates to create what is referred to as a “pre-fabricated panel”, and any combination thereof. Additionally or alternatively, the insulation material can be at least partially constructed from Composite foams, these are acoustical foams that are made by layering different facings or foams together to create enhanced performance for specific application types. Composite foams can meet more than one acoustical requirements at the same time such as providing both sound blocking and sound absorbing capabilities. These can be open or closed cell foams. Additionally or alternatively all the aforementioned materials can be at least partly porous. Additionally or alternatively, all the aforementioned materials can be combined with fire resistant materials.

The term “resonators” interchangeably refers herein to a structure configured to typically act to absorb sound in a narrow frequency range. Resonators include some perforated materials and materials that have openings (holes and slots). Such as a Helmholtz resonator, which has the shape of a bottle. The resonant frequency is governed by the size of the opening, the length of the neck and the volume of air trapped in the chamber. Typically, perforated materials only absorb the mid-frequency range unless special care is taken in designing the facing to be as acoustically transparent as possible.

The term “Bass Traps” interchangeably refers herein to acoustic energy absorbers which are designed to damp low frequency sound energy with the goal of attaining a flatter low frequency (LF) room response by reducing LF resonances in rooms. Similar to other acoustically absorptive devices, they function by turning sound energy into heat through friction. There are generally two types of bass traps: resonating absorbers and porous absorbers. By their nature resonating absorbers tend toward narrow band action [absorb only a narrow range of sound frequencies] and porous absorbers tend toward broadband action [absorbing sound all the way across the audible band—low, mid, and high frequencies], though both types can be altered to be either more narrow, or more broad in their absorptive action. Examples of resonating type bass traps include Helmholtz resonators, and devices based on diaphragmic elements or membranes which are free to vibrate in sympathy with the room's air when sound occurs. Resonating type bass traps achieve absorption of sound by sympathetic vibration of some free element of the device with the air volume of the room. Such free elements in a resonating device can come in many forms such as the air volume captured inside a Helmholtz resonator—or a thin wooden panel held only by its edges [frequently called: “panel absorbers”, a style of diaphragmic absorber]. Resonating absorbers can be made from just about any material that can either form a stiff walled vessel [a glass bottle for example] or any membrane stiff enough to be susceptible to being induced to vibrations by impinging sound.

It is in the scope of the invention wherein the term “diffusion” refers to the efficacy by which sound energy is spread evenly in a given environment. A perfectly diffusive sound space is, as defined in Wikipedia, one that has certain key acoustic properties which are the same anywhere in the space. A non-diffuse sound space would have considerably different reverberation time as the listener moved around the room. Virtually all spaces are non-diffuse. Spaces which are highly non-diffuse are ones where the acoustic absorption is unevenly distributed around the space, or where two different acoustic volumes are coupled. The diffusiveness of a sound field can be measured by taking reverberation time measurements at a large number of points in the room, then taking the standard deviation on these decay times. Alternately, the spatial distribution of the sound can be examined. Small sound spaces generally have very poor diffusion characteristics at low frequencies due to room modes.

Still in the scope of the invention, “diffusors”, and “diffusers” are interchangeably used herein to define means to treat sound aberrations within a medical device, such as echoes. As depicted in Wikipedia, diffusers are an excellent alternative or complement to sound absorption because they do not remove sound energy, but can be used to effectively reduce distinct echoes and reflections while still leaving a live sounding space. Compared to a reflective surface, which will cause most of the energy to be reflected off at an angle equal to the angle of incidence, a diffusor will cause the sound energy to be radiated in many directions, hence leading to a more diffusive acoustic space. It is also important that a diffusor spreads reflections in time as well as spatially. Diffusors can aid sound diffusion, but this is not why they are used in many cases; they are more often used to remove coloration and echoes. The term ‘diffusers’ also relates to MLS Diffusors, 1000 Hz Quadratic-Residue Diffusor, Primitive-Root Diffusors, Optimized Diffusors, Two Dimensional (“Hemispherical”) Diffusors etc.

The term “sound baffle” interchangeably refers herein to a construction or device which reduces the strength (level) of airborne sound, as measured in dB (decibels). Sound baffles are a fundamental tool of noise mitigation, for the practice of minimizing noise or reverberation. An important type of sound baffle is a noise barrier/sound shield. Sound baffles are also applied to walls and ceilings in building interiors to absorb sound energy and thus lessen reverberation. These include, as non-limiting examples, wave baffles, fabric coated baffles, curtain baffles, panel baffles and etc.

Active sound controlling devices that create destructive interferences using a secondary source of noise such as using actuator loudspeakers. Some active sound controlling devices use active feedback mechanisms utilizing information received from sound sensors in various locations, and respond to the specific frequency and sound level received. An active sound control mechanism can be efficiently employed in a system whose generated sound frequency can be calculated.

The term “sound masking” interchangeably refers herein to the addition of natural or artificial sound (such as white noise or pink noise) into an environment to cover up unwanted sound by using auditory masking. As depicted in Wikipedia, this is in contrast to the technique of active noise control. Sound masking reduces or eliminates awareness of pre-existing sounds in a given area and can make a work environment more comfortable, while creating speech privacy so workers can better concentrate and be more productive. Sound masking can also be used in the outdoors to restore a more natural ambient environment. Sound masking is a similar process of covering a distracting sound with a more soothing or less intrusive sound. The masking must reduce the difference between the steady background level and the transient levels associated with both speech and other sounds. Motivation and productivity are improved when this is accomplished. The masking sound itself must not change rapidly and should be as meaningless as possible. As a non-limiting example, masking can be obtained by the generation of an acoustic noise signal such as: white noise, pink noise, blue noise, gray noise, brownian noise, violet noise, a repetitive noise derived in nature (such as the sound of waves), music, speech, and any combination thereof. Additionally or alternatively, the noise signal can be repeated over a predefined amount of time or be administered intermittently, continuously, or in any pattern or combination of the different kinds.

Hybrid sound attenuating systems that employ both active and passive elements to achieve sound reduction and adaptive-passive systems that use passive devices whose parameters can be varied in order to achieve optimal noise attenuation over a band of operating frequencies, such as a tunable Helmholtz resonator. As a non-limiting example, disclosed in the art is “Air transparent soundproof window”, arXiv: 1307.0301 currently available at link: http://phys.org/news/2013-07-materials-scientists-window-mutes-air.html#jCp describing a screen that although passable to air, lowers the sound transmitted by up to 35 dB, by designing specific chambers and holes configured to capture and attenuate sound, consisting of a three-dimensional array of diffraction-type resonators with many holes centered at each individual resonator. Further, the researchers note that changing the size of the hole allows for muting different frequencies.

It is further within the scope of the invention incubator, comprising envelope with air flow openings comprising at least one resonator configured to attenuate sound. Additionally or alternatively, the envelope comprises volume having height represented by h, and is measured preferably in millimeters. The value of h can be constant or variable throughout the medical device. In at least a portion of this volume resonators and attenuators can be implemented. Further this volume can be filled with sound absorptive material situated around the perforations.

The term “sound shield” refers herein after to any sound barriers or sound reflection panel, sound absorbing panel, screens, baffle, or any combination thereof, single or a plurality of, configured to lowering the sound reaching the patient.

The term “reverberation” interchangeably refers herein to a prolongation of the sound in the room caused by continued multiple reflections is called reverberation. This can happen in an at least partially enclosed space during the time it takes a sound to become inaudible and stop emitting energy. When room surfaces are highly reflective, sound continues to reflect or reverberate. The effect of this condition is described as a live space with a long reverberation time. A high reverberation time will cause a build-up of the noise level in a space.

The term “reflections” interchangeably refers herein to a phenomenon that sound reflects back from at least one surface or object before reaching the receiver. These reflections can have unwanted or even disastrous consequences. Reflective corners or peaked ceilings can create a “megaphone” effect potentially causing annoying reflections and loud spaces. Reflective parallel surfaces lend themselves to a unique acoustical problem called standing waves, creating a “fluttering” of sound between the two surfaces. The standing waves can produce natural resonances that can be heard as a pleasant sensation or an annoying one. Reflections can be attributed to the shape of the space as well as the material on the surfaces. Domes and concave surfaces cause reflections to be focused rather than dispersed which can cause annoying sound reflections. Absorptive surface treatments can help to eliminate both reverberation and reflection problems.

The terms “NRC” and “Noise Reduction Coefficient” interchangeably refer herein to a characteristic of a material/product presenting the average absorption across four octave band center frequencies. (250 Hz, 500 Hz, 1000 Hz, 2000 Hz.). It can be roughly estimate that a product with an NRC 0.75 will absorb about 75% of the sound energy that hits it. The highest level is NRC 1.0. Substantially this is the average of the mid frequency absorption rate, rounded to the near 5%, and does not include the high and low frequencies.

The terms “STC” and “Sound Transmission Class” interchangeably refer herein to a number rating of the transmission loss properties of a material and/or product. It is a single-number rating of a material's or an assembly's ability to resist airborne sound transfer at the frequencies 125-4000 Hz. Substantially, this refers to a material's barrier ability qualities. In general, a material and/or product with higher STC rating blocks more noise from transmitting through a partition. STC is highly dependent on the construction of the partition. A partition's STC can be increased by adding mass, increasing or adding air space, adding absorptive material within the partition, and likewise. A partition is given an STC rating by measuring its Transmission Loss over a range of 16 different frequencies between 125-4000 Hz. The STC rating does not assess the low frequency sound transfer. Doors, windows, walls, floors, etc. are tested to determine how much noise passes through.

The term “about” interchangeably refers herein to a divergence of up to plus or minus 20% around a given value.

### Passive Measures to Ensure Correct Noise Levels and Correct Temperature

Insulated glazing (IG), more commonly known as double glazing (or double-pane, and increasingly triple glazing/pane), are double or triple glass window panes separated by an air or other gas filled space to reduce heat transfer across a part of the building envelope. Insulated Glass Units (IGUs) are widely commercialized in various household applications, in the vehicle industry etc., nevertheless, it use in medical applications is still not so common.

It is in the scope of the invention wherein an incubator, incubator's wall or a portion thereof comprises or is in connection with one or more IGUs, e.g., IGUs that are manufactured with glass in range of thickness from e.g., 3 mm to e.g. 10 mm or more in special applications.

It is further in the scope of the invention wherein the glass panes are separated by one or more “spacers”. A spacer, according to an embodiment of the invention, is the piece that separates the two panes of glass in an insulating glass system, and seals the gas space between them. It is further in the scope of the invention wherein spacers are made primarily of metal and fiber, which manufacturers thought provided more durability. However, metal spacers conduct heat (unless the metal is thermally improved), undermining the ability of the IGU to reduce heat flow. It may also result in water or ice forming at the bottom of the sealed unit because of the sharp temperature difference between the window and the surrounding air. To reduce heat transfer through the spacer and increase overall thermal performance, and thus it is further in the scope of the invention wherein the spacer is made of a less-conductive material such as structural foam. It is further in the scope of the invention wherein the spacer is made of aluminum that also contains a highly structural thermal barrier reduces condensation on the glass surface and improves insulation, as measured by the overall U-factor. It is further in the scope of the invention wherein the spacer which is used is useful for reducing heat flow in glazing configurations and further has characteristics for sound dampening where external noise is an issue.

It is further in the scope of the invention wherein the spacers are filled with or contain desiccant to remove moisture trapped in the gas space during manufacturing, thereby lowering the dew point of the gas in that space, and preventing condensation from forming on the second surface when the outside glass pane temperature falls.

It is further in the scope of the invention wherein the IGUs are set useful for avoiding heat loss from traditional spacer bars, and discloses use of long-term-durability of improved metal (aluminum with a thermal barrier) and foam spacers. Materials which can be used for double glazing include aluminum, PVC, and wood (timber).

### Thermal Performance

The maximum insulating efficiency of a standard IGU is determined by the thickness of the space containing the gas. Too little space between the panes of glass results in heat loss by diffusion between the panes (heat from one pane travelling through the fill gas the other pane) while if too large a gap is used, convection currents are not damped out by the gas viscosity and transfer heat between the panel. It is further in the scope of the invention wherein the sealed units achieve maximum insulating values using a gas space of 16-19 mm (0.63-0.75 in) when measured at the center of the IGU. When combined with the thickness of the glass panes being used, this can result in an overall thickness of the IGU of 22-25 mm (0.87-0.98 in) for 3 mm (0.12 in) glass to 28-31 mm (1.1-1.2 in) for 6.35 mm (0.250 in) plate glass.

It is further in the scope of the invention wherein IGU's thickness is a compromise between maximizing insulating value and the ability of the framing system used to carry the unit. Some residential and most commercial glazing systems can accommodate the ideal thickness of a double paned unit. Issues arise with the use of triple glazing to further reduce heat loss in an IGU. The combination of thickness and weight results in units that are too unwieldy for most residential or commercial glazing systems, particularly if these panels are contained in moving frames or sashes.

It is further in the scope of the invention wherein this trade-off does not apply to Vacuum Insulated Glass (VIG), or evacuated glazing, as heat loss due to convection is eliminated, leaving radiation losses and conduction through the edge seal. These VIG units have most of the air removed from the space between the panes, leaving a nearly-complete vacuum. VIG units which are currently on the market are hermetically sealed along their perimeter with solder glass, that is, a glass frit having a reduced melting point. Such a glass seal is rigid, and will experience increasing stress with increasing temperature differential across the unit. This stress may prevent Vacuum glazing from being used when the temperature differential is too great. One manufacturer provides a recommendation of 35° C.

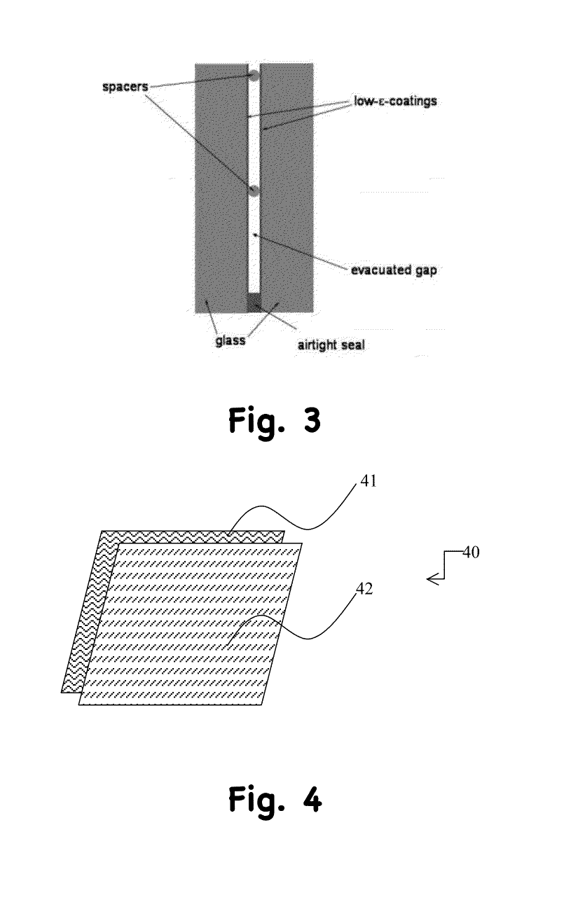

It is further in the scope of the invention wherein isolated incubators are characterized by U-values for walls of 0.3 W/(m²K) or even below can be realized. In such incubators, glazings with typical U-values of 1.0 W/(m²K) or higher are thermal weak spots in the façade. One attractive possibility to essentially improve the insulation properties of a glazing is to evacuate the space between the glass panes. This virtually eliminates heat transport due to conduction and convection of the filling gas. The glass panes can be prevented from collapsing by using a matrix of spacers (FIG. 3, see Weinläder et al., WIG, currently available link which is incorporated herein as a reference: http://www.bine.info/fileadmin/content/Publikationen/Englische_Infos/projekt_{—}0108_engl_internetx.pdf). Evacuated glazings are already available as a commercial product however have U-values of 1.1 W/(m²K) or higher.

The unit of measurement for the heat loss through a component: the U-value of glazing, is a parameter characterizing the heat transmittance through the central area of the glazing, i.e. without edge effects, and stating the stationary heat flow density for each temperature difference between the ambient temperatures on each side. The U-value is stated in Watts per square meter and Kelvin (W/m2K). The lower the Uvalue, the better the thermal insulation. The unit of measurement is W/m2K. U-value of glazing: U_{g} (=‘U_{glass}’); U-value of window: U_{w} (=‘U_{window}’); U-value of frame: U_{f} (=‘U_{frame}’); U-value for curtain walls: U_{cw} (‘U_{courtain-wall}’); Ψ-value=linear heat transmittance coefficient (PSI); U_{g}-value: The basis for calculating the U_{g} value is EN 673 in the European Standards.

The nominal U_{g} value of glazing depends on four factors: emissivity of the function layer, width of the pane cavity, the type of gas filling and the degree of gas filling. To ascertain the values for assessment, national regulations must be complied with.

### U_{f}-Value

The heat transmittance coefficient of the frame section Uf is usually determined by measurement of the entire section according to national regulations. The Uf-value can however also be calculated using a Finite-Element Program (FEM) or Finite-Difference program.

### Ψ-Value

The linear heat transmittance coefficient Ψ for the window describes the thermal bridge in the transition area between the window frame and the insulating glass edge. The Ψ-value is the figure for the heat quantity lost per unit of time through 1 m of the section line with 1 K of temperature difference between the room side and the outside. The unit of measurement is W/mK.

The linear heat transmittance coefficients needed for Uw-value calculation can be taken as fixed values from the tables given by national regulations. Usually, Ψ-values are provided by the manufacturers of spacer sections for standard frame materials such as metal, wood or plastic. Data sheets for different systems and frame materials are provided by the suppliers.

### U_{w}-Value

The nominal value U_{w} of the heat transmittance coefficient is either read off from the appropriate tables, or calculating using the following Formula I:

$${U}_{w} = \frac{{A}_{f} + {U}_{f} + {A}_{g} + {U}_{g} + \sum ( {I}_{g} * Ψ )}{{A}_{f} + {A}_{g}}$$

Where U_{w}: heat transmittance of the window; U_{f}: heat transmittance of the frame (measured value!); U_{g}: heat transmittance of the glazing (nominal value); A_{f}: area of the frame; A_{g}: Area of the glass; I_{g}: area of the glazing; Ψ: linear heat transmittance of the glass edge.

It is in the scope of the invention wherein the IGU heat insulation is determined by the U_{w} value.

It is further in the scope of the invention wherein the vacuum technology is also used in non-transparent insulation products, e.g., vacuum insulated panels.

An older-established way to improve insulation performance is to replace air in the space with a lower thermal conductivity gas. Gas convective heat transfer is a function of viscosity and specific heat. It is further in the scope of the invention wherein monatomic gases such as argon, krypton and xenon are used since (at normal temperatures) they do not carry heat in rotational modes, resulting in a lower heat capacity than poly-atomic gases. Argon has a thermal conductivity 67% that of air, krypton has about half the conductivity of argon. Krypton and Xenon are very expensive. These gases are used because they are non-toxic, clear, odorless, chemically inert, and commercially available because of their widespread application in industry. Some manufacturers also offer sulfur hexafluoride as an insulating gas, especially to insulate sound. It has only ⅔ the conductivity of argon, but it is stable, inexpensive and dense. However, sulfur hexafluoride is an extremely potent greenhouse gas that contributes to global warming. In Europe, SF6 falls under the F-Gas directive which ban or control its usage for several applications. Since 1 Jan. 2006, SF6 is banned as a tracer gas and in all applications except high-voltage switchgear.

In general, the more effective a fill gas is at its optimum thickness, the thinner the optimum thickness is. For example, the optimum thickness for krypton is lower than for argon, and lower for argon than for air. However, since it is difficult to determine whether the gas in an IGU has become mixed with air at time of manufacture (or becomes mixed with air once installed), many designers prefer to use thicker gaps than would be optimum for the fill gas if it were pure. It is further in the scope of the invention wherein Argon is used in insulated incubator's glazing as it is the most affordable.

### Heat Insulating Properties

The effectiveness of insulated glass can be expressed as an R-value. The higher the R-value, the greater is its resistance to heat transfer. A standard IGU consisting of clear uncoated panes of glass (or lites) with air in the cavity between the lites typically has an R-value of 0.35 K·m2/W. Using US customary units, a rule of thumb in standard IGU construction is that each change in the component of the IGU results in an increase of 1 R-value to the efficiency of the unit. Adding Argon gas increases the efficiency to about R-3. Using low emissivity glass on second surface will add another R-value. Properly designed triple glazed IGUs with low emissivity coatings on second and forth surfaces and filled with argon gas in the cavities result in IG units with R-values as high as R-5. Certain vacuum insulated glass units (VIG) or multi-chambered IG units using coated plastic films result in R-values as high as R-12.5

Additional layers of glazing provide the opportunity for improved insulation. While the standard double glazing is most widely used, triple glazing is not uncommon, and quadruple glazing is produced for very cold environments such as Alaska. Even quintuple glazing (four cavities, five panes) is available—with mid-pane insulation factors equivalent to walls.

### R-Value is the Reciprocal of U-Factor.

Thermal transmittance, also known as U-value (U-factor), is the rate of transfer of heat (in watts) through one square metre of a structure divided by the difference in temperature across the structure. It is expressed in watts per metres squared kelvin, or W/m²K. Well-insulated parts of a building have a low thermal transmittance whereas poorly insulated parts of a building have a high thermal transmittance. Losses due to thermal radiation, thermal convection and thermal conduction are taken into account in the U-value:

Φ=A×U×(T1−T2)

where Φ is the heat transfer in watts, U is the thermal transmittance, T1 is the temperature on one side of the structure, T2 is the temperature on the other side of the structure and A is the area in square meters.

Thermal transmittances of most walls and roofs can be calculated using ISO 6946, unless there is metal bridging the insulation in which case it can be calculated using ISO 10211. For most ground floors it can be calculated using ISO 13370. For most windows the thermal transmittance can be calculated using ISO 10077 or ISO 15099. ISO 9869 describes how to measure the thermal transmittance of a structure experimentally.

Typical thermal transmittance values for common building structures are as follows: single glazing: 5.7 W/m²K; single glazed windows, allowing for frames: 4.5 W/m²·K; double glazed windows, allowing for frames: 3.3 W/m²·K; double glazed windows with advanced coatings: 2.2 W/m²·K; double glazed windows with advanced coatings and frames: 1.2 W/m²·K; triple glazed windows, allowing for frames: 1.8 W/m²·K; triple glazed windows, with advanced coatings and frames: 0.8 W/m²·K; well-insulated roofs: 0.15 W/m²·K; poorly insulated roofs: 1.0 W/m²·K; well-insulated walls: 0.25 W/m²·K; poorly insulated walls: 1.5 W/m²·K; well insulated floors: 0.2 W/m²·K; poorly insulated floors: 1.0 W/m²·K.

In practice the thermal transmittance is strongly affected by the quality of workmanship and if insulation is fitted poorly, the thermal transmittance can be considerably higher than if insulation is fitted well.

### Acoustic Insulating Properties

It is further in the scope of the invention wherein the incubator's wall and insulation thereof is in reference to noise mitigation. In these circumstances a large air space improves the noise insulation quality or Sound transmission class. It is further in the scope of the invention wherein asymmetric double glazing is used. Hence, using different thicknesses of glass rather than the conventional symmetrical systems improve the acoustic attenuation properties of the IGU and reduce noise within the incubator. It is further in the scope of the invention wherein standard air spaces are used, and sulfur hexafluoride is further used to replace or augment an inert gas and improve acoustical attenuation performance.

Other glazing material variations affect acoustics. It is further in the scope of the invention wherein the glazing configurations for sound dampening include laminated glass with varied thickness of the interlayer and thickness of the glass. Including a structural, thermally improved aluminum thermal barrier air spacer in the insulating glass can improve acoustical performance by reducing the transmission of exterior noise sources in the fenestration system.

### Estimating Noise Levels

### Measurement Indices

### Sound Transmission Loss (STL)

The average sound transmission loss (STL) is useful for determining the effectiveness of glazed panels to isolate exterior noise (such as traffic) from a building. It is derived from the average of the measured transmission loss at eighteen ⅓ octave frequency bands between 100 Hz and 5000 Hz. The average STL is measured in decibels, the higher the average STL dB figure, the more effective the glazing will be in reducing sound transmission.

### Sound Transmission Class (STC)

The sound transmission class (STC) is useful for determining the noise reduction offered by internal building elements such as partitions and walls. It is a measure that relates the sound reduction performance against sounds which normally occur inside a building (such as voices, telephones, music) The STC is a numerical class rating and cannot be compared with the STL. The STC is derived from a best fit curve comparison of a reference STC curve to the insulation curve. The higher the STC the higher the overall sound reduction.

### Mean Sound Reduction Index (Rₘ)

The mean sound reduction index has traditionally been considered one of the more complete methods of comparing insulation performance of glass. Mean reduction establishes a level of performance over a wide range of frequencies. This average of insulation values, measured in dB, is a simple indicator of a products sound insulation performance.

### Weighted Sound Reduction Index (R_{W})

The weighted sound reduction index is now the most common index used internationally since its adoption in the BS and ISO standards. This index has largely replaced the Rm and STC index because the weighted reduction incorporates frequency modified correction for the human ears response. The RW is reported in dB and is a composite rating of sound reduction at frequencies from 100-5000 Hz. Numerically, it is comparable to the STC values but the numbers are in dB.

### Traffic Noise Sound Reduction (Rₜᵣ)

The traffic noise reduction index is distinct from the other indices because of its dependence on a specific offending noise type. The reduction is calculated based upon the offending sound source being a typical sound spectrum of road and traffic sound and intensities. Traffic noise reduction is measured in dBA.

### Apparent Sound Reduction Index (R′)

This is a field measurement which attempts to measure the sound reduction index of a material on a real completed construction (e.g. a wall between two offices, houses or cinema auditoriums). It is unable to isolate or allow for the result of alternate sound transmission routes and therefore will generally produce a lower result than the laboratory measured value. The calculation method used to produce the Sound Reduction Index takes into account the relative size of the tested rooms, and the size of the tested panel, and is therefore (theoretically) independent of these features, therefore a 1×1 panel of plasterboard (drywall) should have the same R_{w} as a 10×10 panel.

### Normalized Level Difference (Dn)

This is an index which is measured in field conditions, between “real” rooms. It is a measurement which deliberately includes effects due to flanking routes and differences in the relative size of the rooms. It attempts however to normalize the measured difference level to the level which would be present when the rooms are furnished by measuring the quantity of acoustic absorption in the receiving room and correcting the difference level to the level which would be expected if there was 10 m² Sabine absorption in the receiving room. Detailed, accurate knowledge of the dimensions of the receiving room are required.

### Standardized Level Difference (D_{nT})

Similar to the normalized level difference, this index corrects the measured difference to a standardized reverberation time. For dwellings the standard reverberation time used is 0.5 seconds, for other larger spaces longer reverberation times will be used. 0.5 seconds is often cited as approximately average for a medium sized, carpeted and furnished living room. Due to not requiring detailed and accurate knowledge of the dimensions of the test rooms, this index is easier to obtain, and arguably of slightly more relevance. Once the difference level or sound reduction index is obtained, the weighted value may be obtained from the corrected spectrum as described above from the reference curve.

The Standardized Level Difference (D_{nT}) is calculated using Formula II:

$${D}_{nT} = D + 10 log \frac{T}{{T}_{0}}$$

Where: D=level difference; T=reverberation time in the receiving room; T0=reference Reverberation Time, 0.5 seconds for dwellings.

It is in the scope of the invention wherein the IGU noise insulation is determined by the D_{nT} value.

Estimating Heat Loss from Double Glazed Windows

Given the thermal properties of the sash, frame, and sill, and the dimensions of the glazing and thermal properties of the glass, the heat transfer rate for a given window and set of conditions can be calculated. This can be calculated in kW (kilowatts), but more usefully for cost benefit calculations can be stated as kWH pa (kilowatt hours per annum), based on the typical conditions over a year for a given location. It is further in the scope of the invention wherein the glass panels in double glazed windows transmit heat in both directions by radiation, across the panes by convection, and by conduction around the perimeter seals. The actual rates will vary with the conditions throughout the year, and while solar gain is much welcomed in the winter, it may result in increased air conditioning costs in the summer. The unwanted heat transfer can be mitigated by for example using curtains in the winter and using sun shades in the summer. In an attempt to provide a useful comparison between alternative window constructions the British Fenestration Rating Council have defined a “Window Energy Rating” WER, ranging from A for the best down through B and C etc. This takes into account a combination of the heat loss through the window (U value, the reciprocal of R-value), the solar gain (g value), and loss through air leakage around the frame (L value). For example, an A Rated window will in a typical year gain as much heat from solar gain as it loses in other ways (however the majority of this gain will occur during the summer months, when the heat may not been needed by the building occupant). This provides the incubator with a better thermal performance than a typical wall.

It is in the scope of the invention wherein the term ‘glass’ refers to any at least partially transparent materials, such as glass, polymers (PMMA) and mixtures thereof.

### Incubator's Canopy Comprising Laminated Glass

A neonatal transport incubator is a device consisting of a portable rigid boxlike enclosure with insulated walls in which an infant may be kept in a controlled environment while being transported for medical care. The device may include straps to secure the infant, a battery-operated heater, an AC-powered battery charger, a fan to circulate the warmed air, a container for water to add humidity, and provision for a portable oxygen bottle, Additionally or alternatively, a neonatal incubator is a device with an enclosure intended to contain a baby and having transparent section(s) which allow(s) for viewing of the baby, and provided with means to control the environment of the baby primarily by heated air within the enclosure (21 CFR Part 880.5410).

A neonatal incubator is a device consisting of a rigid boxlike enclosure in which an infant may be kept in a controlled environment for medical care. The device may include an AC-powered heater, a fan to circulate the warmed air, a container for water to add humidity, a control valve through which oxygen may be added, and access ports for nursing care (21 CFR Part 880.5400).

MRI animal's incubators (MAIs), such as the commercially available M-series instruments animal incubators by Aspect Imaging Ltd, are known in the art to assist in MRI imaging of small and large animals (mice to rabbits), along pre-clinical studies.

The aforesaid neonatal transport incubators, neonatal incubators and MAIs are interchangeably denoted herein as “incubator” or “incubators”.

It is in the scope of the invention wherein at least a portion of the incubator's canopy's glass is laminated. Laminated glass is a type of safety glass that holds together when shattered. In the event of breaking, it is held in place by an interlayer, typically of polyvinyl butyral (PVB), between its two or more layers of glass. The interlayer keeps the layers of glass bonded even when broken, and its high strength prevents the glass from breaking up into large sharp pieces. This produces a characteristic “spider web” cracking pattern when the impact is not enough to completely pierce the glass.

Reference is now made to FIG. 4, illustrating in an out of scale schematic manner a glass (41) laminated by a laminate (42), here PVB to yield with a sound-proof and thermo-regulating laminated glass for incubators.

It is further in the scope of the invention wherein sound reduction improve with increased glass thickness due to the greater mass involved; Sound reduction will decrease somewhat with increasingly larger glass areas but not enough to make much difference in the majority of architectural glass sizes; Sound reduction will improve with the use of laminated glass due to the vibration dampening effect of the PVB interlayer. Laminated glass is particularly effective for interior partitions as it reduces the ‘coincidence dip’ attributed to monolithic glass in the 1000-2000 Hz range, a range attributed to the human voice; Sound reduction will improve with the use of glass/airspace combinations, but the performance is critically dependent upon the width of the airspace. An airspace of 100 mm is generally regarded as a minimum for reasonable benefits at medium to high frequencies. The optimum airspace is about 300 mm.

It is further in the scope of the invention wherein a laminated makeup is about 2.5 mm glass/0.38 mm interlayer/2.5 mm glass. This gives a final product that would be referred to as 5.38 laminated glass. Multiple laminates and thicker glass increases the strength. Bullet-resistant glass is usually constructed using polycarbonate, thermoplastic, and layers of laminated glass. A similar glass is often used in airliners on the front windows, often three sheets of 6 mm toughened glass with thick PVB between them. Newer developments have increased the thermoplastic family for the lamination of glass. Besides PVB, important thermoplastic glass lamination materials today are EVA (ethyl vinyl acetate) and TPU (thermoplastic polyurethane). The adhesion of PVB/TPU and EVA is not only high to glass but also to Polyester (PET) Interlayer. Since 2004 metallized and electroconductive PET-Interlayers are used as substrate for light emitting diodes and laminated to or between glasses.

It is further in the scope of the invention wherein according to an embodiment of the invention, Top Layer is Glass; Interlayer (e.g.,) is Transparent thermoplastic material like TPU, PVB or EVA; Interlayer (e.g.,) is LED (light emitting diodes) on transparent conductive Polymer; Interlayer (e.g.,) is Transparent thermoplastic material like TPU, PVB or EVA; and Bottom layer is Glass.

### Active Measures to Ensure Correct Noise Levels and Correct Temperature

### Sound Masking (White Noise)

In signal processing, white noise is a random signal with a constant power spectral density. The term is used, with this or similar meanings, in many scientific and technical disciplines, including physics, acoustic engineering, telecommunications, statistical forecasting, and many more. White noise refers to a statistical model for signals and signal sources, rather than to any specific signal.

The term is also used for a discrete signal whose samples are regarded as a sequence of serially uncorrelated random variables with zero mean and finite variance. Depending on the context, one may also require that the samples be independent and have the same probability distribution. In particular, if each sample has a normal distribution with zero mean, the signal is said to be Gaussian white noise.

The samples of a white noise signal may be sequential in time, or arranged along one or more spatial dimensions. In digital image processing, the pixels of a white noise image are typically arranged in a rectangular grid, and are assumed to be independent random variables with uniform probability distribution over some interval. The concept can be defined also for signals spread over more complicated domains, such as a sphere or a torus.

An infinite-bandwidth white noise signal is a purely theoretical construction. The bandwidth of white noise is limited in practice by the mechanism of noise generation, by the transmission medium and by finite observation capabilities. Thus, a random signal is considered “white noise” if it is observed to have a flat spectrum over the range of frequencies that is relevant to the context. For an audio signal, for example, the relevant range is the band of audible sound frequencies, between 20 to 20,000 Hz. Such a signal is heard as a hissing sound, resembling the /sh/ sound in “ash”. In music and acoustics, the term “white noise” may be used for any signal that has a similar hissing sound.

It is in the scope of the invention wherein inside the incubator there is the possibility to play white noise at variables levels.

### Active Noise Cancellation

Active noise control (ANC), also known as noise cancellation, or active noise reduction (ANR), is a method for reducing unwanted sound by the addition of a second sound specifically designed to cancel the first.

Sound is a pressure wave, which consists of a compression phase and a rarefaction phase. A noise-cancellation speaker emits a sound wave with the same amplitude but with inverted phase (also known as antiphase) to the original sound. The waves combine to form a new wave, in a process called interference, and effectively cancel each other out—an effect which is called phase cancellation.

Modern active noise control is generally achieved through the use of analog circuits or digital signal processing. Adaptive algorithms are designed to analyze the waveform of the background aural or non-aural noise, then based on the specific algorithm generate a signal that will either phase shift or invert the polarity of the original signal. This inverted signal (in antiphase) is then amplified and a transducer creates a sound wave directly proportional to the amplitude of the original waveform, creating destructive interference. This effectively reduces the volume of the perceivable noise.

A noise-cancellation speaker may be co-located with the sound source to be attenuated. In this case it must have the same audio power level as the source of the unwanted sound. Alternatively, the transducer emitting the cancellation signal may be located at the location where sound attenuation is wanted (e.g. the user's ear). This requires a much lower power level for cancellation but is effective only for a single user. Noise cancellation at other locations is more difficult as the three-dimensional wavefronts of the unwanted sound and the cancellation signal could match and create alternating zones of constructive and destructive interference, reducing noise in some spots while doubling noise in others. In small enclosed spaces (e.g. inside an incubator) global noise reduction can be achieved via multiple speakers and feedback microphones, and measurement of the modal responses of the enclosure.

Applications can be “1-dimensional” or 3-dimensional, depending on the type of zone to protect. Periodic sounds, even complex ones, are easier to cancel than random sounds due to the repetition in the wave form.

Protection of a “1-dimension zone” is easier and requires only one or two microphones and speakers to be effective. Several commercial applications have been successful: noise-cancelling headphones, active mufflers, and the control of noise in air conditioning ducts. The term “1-dimension” refers to a simple pistonic relationship between the noise and the active speaker (mechanical noise reduction) or between the active speaker and the listener (headphones).

Protection of a 3-dimension zone requires many microphones and speakers, making it more expensive. Each of the speakers tends to interfere with nearby speakers, reducing the system's overall performance. Noise reduction is more easily achieved with a single listener remaining stationary but if there are multiple listeners or if the single listener turns his head or moves throughout the space then the noise reduction challenge is made much more difficult. High frequency waves are difficult to reduce in three dimensions due to their relatively short audio wavelength in air. The wavelength in air of sinusoidal noise at approximately 800 Hz is double the distance of the average person's left ear to the right ear; such a noise coming directly from the front will be easily reduced by an active system but coming from the side will tend to cancel at one ear while being reinforced at the other, making the noise louder, not softer. High frequency sounds above 1000 Hz tend to cancel and reinforce unpredictably from many directions. In sum, the most effective noise reduction in three-dimensional space involves low frequency sounds. Commercial applications of 3-D noise reduction include the protection of aircraft cabins and car interiors, but in these situations, protection is mainly limited to the cancellation of repetitive (or periodic) noise such as engine-, propeller- or rotor-induced noise. This is because an engine's cyclic nature makes fast Fourier transform analysis and the noise cancellation easier to apply.

It is further in the scope of the invention wherein a noise cancelling system is part of the incubator. A number of sound sensors, or microphones, are located inside the incubator and perform constant monitoring of the noise level inside the incubator. A series of speakers will transmit an anti-wave that will cancel all the noises above the recommended level of 45 dB.

### Active Thermoregulation of the Incubator

Thermo-regulation is carried out by a temperature regulating mechanism located at the end of the incubator. The TRM introduces thermoregulated air to the container in a quiet and gentle flow parallel to the longitudinal axis of the incubator. The desired temperature is set by the user using a specific interface.

The term “thermo-regulated environment” refers hereinafter to an environment that its air temperature is in a constant pre-determined temperature with an error of ±0.5° C.

The term “heating/cooling module” refers hereinafter to a module that controls the temperature either by heating or by cooling or by doing both. More specifically the term relates to an air conditioned system, an infrared heater, a water/oil-heated radiator, a coiled heater, an open coil air heater, a round open coil air heater, a convection heater, straight or formed tubular heaters, a quartz tube air heater, a capacitor-type heater, a Pelletier module, etc.

The TMR receives information from thermo-sensors inside the incubator, and also possible, from a sensor attached to the newborn.

It is further in the scope of the invention wherein the incubator comprises a temperature regulating mechanism comprising thermo-sensors in the incubator and/or the newborn, a human interface unit for setting the desired temperature and a heating/cooling module.

What is claimed is:

1. An incubator comprising at least one canopy, said canopy comprising at least one insulated glass unit (IGU, 10); said IGU comprising at least one first inner glass (1a) and at least one second external glass (1b), said inner and external glasses are spaced by a spacer (3); said inner glass is characterized by thickness W_{inner glass}; said external glass is characterized by thickness W_{external glass}; said glasses separate between at least three different atmospheres: a first atmosphere (21) within said canopy; a second atmosphere (22) between said inner and external glasses; and, a third atmosphere (23) outside said canopy; said second atmosphere (22) comprises a gas; said IGU is characterized by U-factor value U (W/m²K) and a Noise Standardized Level Difference (DnT) (dB); said IGU is adapted to provide said canopy with sound and thermal insulation by means of at least one element selected from a group consisting of (a) said W_{inner glass}; (b) said W_{external glass}; (c) said U-factor value, (d) said Noise Standardized Level Difference (DnT); and any combination thereof; and at least one vacuum mechanism (VIG), adapted to provide vacuum into the space between said inner and external panels; wherein said U-factor and D_{nT} are provided according to Formula I and Formula II, such that said IGU is configured to (i) avoid sound pressure levels in said inner atmosphere from exceeding 45 dB, and (ii) to increase temperature regulation in said inner atmosphere, such that temperature interval in not greater than 1° C. for air controlled transport incubators and not greater than 0.5° C. for baby controlled transport incubators.

2. The incubator of claim 1, wherein said IGU comprising a gas space of about 16 to about 19 mm when measured at the center of the IGU.

3. The incubator of claim 1, wherein said IGU is characterized by an overall thickness of the IGU of about 22 to about 25 mm for 3 mm glass to about 28 to about 31 mm for 6.35 mm plate glass.

4. The incubator of claim 1, wherein said atmosphere between said first and second glasses is filled with gases selected from a group consisting of: argon, krypton, xenon, sulfur hexafluoride and any combination thereof.

5. The incubator of claim 1, wherein said IGU is a Vacuum Insulated Glass (VIG) comprising an evacuating gap.

6. The incubator of claim 1, wherein said VIG is characterized by U-factor values for walls of about 0.3 to about 1.2 W/(m²K).

7. The incubator of claim 1, wherein said IGU is selected from a group consisting of laminated glass, IGU, VIG and a combination thereof.

8. The incubator of claim 1, characterized by a laminate which reduces the ‘coincidence dip’ attributed to monolithic glass in the range about 1000 to about 2000 Hz range.

9. The incubator of claim 1, wherein said temperature control mechanism consisting by a group consisting of: air conditioned system, an infrared heater, a water/oil-heated radiator, a coiled heater, an open coil air heater, a round open coil air heater, a convection heater, straight or formed tubular heaters, a quartz tube air heater, a capacitor-type heater, a Pelletier module.

10. The incubator of claim 1, comprises a reactive acoustical device, comprising a microphone, noise cancel circuitry, speakers and connection to a power source; configured to cancel said device's surrounding acoustical noise by destructive interference.

11. A method of avoiding sound pressure levels in an inner atmosphere of an incubator from exceeding 45 dB, and increasing temperature regulation in said inner atmosphere, such that temperature interval in not greater than 1° C. for air controlled transport incubators and not greater than 0.5° C. for baby controlled transport incubators, comprising a step of providing an incubator with a canopy, and further providing said canopy with at least one insulated glass unit (IGU, 10); further providing said IGU with at least one first inner glass (1a) and at least one second external glass (1b), said inner and external glasses are spaced by a spacer (3).

12. The method of claim 11, further comprising step of providing said IGU with a gas space of about 16 to about 19 mm when measured at the center of the IGU.

13. The method of claim 11, further comprising step of providing said IGU with an overall thickness of about 22 to about 25 mm for 3 mm glass to about 28 to about 31 mm for 6.35 mm plate glass.

14. The method of claim 11, further comprising step of providing said atmosphere within said first and second glasses is filled with gases selected from a group consisting of: argon, krypton, xenon, sulfur hexafluoride and any combination thereof.

15. The method of claim 11, further comprising step of providing said IGU with Vacuum Insulated Glass (VIG) comprising an evacuating gap.

16. The method of claim 11, wherein said VIG is characterized by U-values for walls of about 0.3 to 1.2 W/(m²K).

17. A method of sound dampening in an incubator comprising a step of selecting said IGU from a group consisting of laminated glass, IGU, VIG and a combination thereof.

18. The method of claim 17, further comprising step of reducing the ‘coincidence dip’ attributed to monolithic glass in the range of about 1000 to about 2000 Hz.

19. The method of claim 17, further comprising step of providing said temperature control mechanism consisting by a group consisting of: air conditioned system, an infrared heater, a water in oil-heated radiator, a coiled heater, an open coil air heater, a round open coil air heater, a convection heater, straight or formed tubular heaters, a quartz tube air heater, a capacitor-type heater, a Pelletier module.

20. The method of claim 17, further comprising step of providing a reactive acoustical device, comprising a microphone, noise cancel circuitry, speakers and connection to a power source; configured to cancel said device's surrounding acoustical noise by destructive interference.
